(19) United States
(12) Patent Application Publication
(10) Pub. No.: US 2006/0142733 A1
(43) Pub. Date: Jun. 29, 2006

(54) Catheter tip and method of attaching a catheter tip to a catheter shaft

(76) Inventor: Andrew Forsberg, Minneapolis, MN (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(21) Appl. No.: 11/020,759

(22) Filed: Dec. 23, 2004

(51) Int. Cl. A61M 25/00

(52) U.S. Cl. 604/523; 264/138; 264/259

(57) ABSTRACT

The present invention is a method of bonding a shaft tip to a distal end of a catheter or sheath shaft. The method includes extruding a shaft to have an initial outside diameter that is greater than an outside diameter the shaft will have when finished. A lap joint area is then ground into the distal end of the shaft. The tip is then insert molded over the lap joint area. The tip includes a collar at a proximal end of the tip that provides additional thermal mass to facilitate the bonding of the tip to the shaft. The shaft is then ground from its initial outside diameter to its finished outside diameter.

### FIELD OF THE INVENTION

The present invention relates to catheters and sheaths and methods of making catheters and sheaths. More particularly, the present invention relates to tips for the shafts of catheters and sheaths and methods of attaching tips to such shafts.

### BACKGROUND OF THE INVENTION

There is a need for catheter and sheath shafts having complex tip geometries. In the past, such shafts were produced by thermoforming the shaft tip in several discrete operations. Unfortunately, this is labor intensive and results in an unacceptably high scrap rate.

In an effort to find a replacement process for thermoforming, the inventor of the present invention attempted to injection mold the tip separately and then bond the tip to the end of a shaft. However, this often resulted in inadequate bond strength between the injection molded tip and the end of the shaft.

In another effort, the inventor of the present invention also attempted to insert mold the shaft tip directly to the end of a shaft. Unfortunately, this process also provided less than desirable results. For instance, the tip material, when injected into the mold, has very little thermal mass and, as a result, often does not contain enough energy to adequately melt the shaft material to allow sufficient mixing between the two materials to form a sufficiently strong bond.

Another problem with insert molding is that the shutoff between the mold steel and the shaft is difficult to achieve. Without high clamping pressures between the mold steel and the outer surface of the shaft, flash will flow past the edge of the mold and proximally along the distal surface of the shaft. Often this flash will erode or otherwise deform the outer distal surface of the shaft, resulting in scrap. Unfortunately, high clamping pressures tend to crimp or otherwise deform the distal end of the shaft. This reduces the surface area available for the tip to bond to the shaft and results in inadequate bond strength and scrap. Additionally, it also creates an imperfection in the surface finish that may affect shaft functionality.

There is a need in the art for a shaft tip that facilitates its bonding to a catheter or sheath shaft with less labor and less scrap. There is also a need in the art for a method of manufacturing shafts that allows a tip to be bonded to a shaft with less labor and resulting scrap. There is also a need in the art for a catheter or sheath shaft made by such a method of manufacturing.

### BRIEF SUMMARY OF THE INVENTION

The present invention, in one embodiment, is a shaft tip for bonding to a distal end of a catheter or sheath shaft. The tip includes a generally cylindrical first portion and a collar portion. The generally cylindrical first portion includes a first outside diameter, a proximal end adapted to bond to the distal end of the shaft, and a distal end opposite the proximal end. The collar portion extends around a proximal portion of the first portion and includes a second outside diameter that is greater than the first outside diameter.

In one embodiment, the distal end of the shaft has a lap joint area and the tip is adapted to be bonded over the lap joint area. In one embodiment, the collar portion is adapted to abut against, and bond with, a lap joint face. In one embodiment, the second outside diameter is less than an extruded outside diameter of the shaft.

The present invention, in one embodiment, is a method of bonding a shaft tip to a distal end of a catheter or sheath shaft. The method includes providing a shaft with an initial outside diameter and then forming a lap joint area in the distal end of the shaft. The method also includes molding the tip over the lap joint area. The tip includes a collar at a proximal end of the tip. Finally, the method includes reducing the initial outside diameter of the shaft down to a finished outside diameter.

In one embodiment of the method, the collar has an outside diameter that is greater than the finished outside diameter. In one embodiment, the outside diameter of the collar is also less than the initial outside diameter of the shaft.

In one embodiment, the method further includes forming a point of contact between a mold face and a lap face of the lap joint area such that the point of contact is outside the finished outside diameter. In one embodiment, the tip is molded over the lap joint area via an insert molding process.

In one embodiment of the method, the initial outside diameter is reduced down to the finished outside diameter by a grinding process. In one embodiment, this is achieved via a centerless grinder.

The present invention, in one embodiment, is a catheter or sheath shaft made according to the aforementioned method.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a flow chart outlining the procedures comprising a method of attaching or bonding a shaft tip to a catheter or sheath shaft.

FIG. 2 is a longitudinal sectional elevation of a distal end of a shaft that was extruded to have an outside diameter that is larger than what will be the shaft's finished outside diameter.

FIG. 3 is the same view of the distal end of the shaft depicted in FIG. 2, except a lap joint has been formed in the shaft.

FIG. 4 is the same view of the distal end of the shaft depicted in FIG. 3, except a mold has been placed over the lap joint area of the shaft.

FIG. 5 is the same view of the distal end of the shaft depicted in FIG. 4, except a tip with a collar portion has been insert molded onto the lap joint area of the shaft.

FIG. 6 is the same view of the distal end of the shaft depicted in FIG. 5, except the mold has been removed and the shaft now has a tip with a collar portion.

FIG. 7 is the same view of the distal end of the shaft depicted in FIG. 6, except the collar portion of the tip and the excess portion of the shaft sidewall has been ground away such that the shaft now has its finished outside diameter.

FIG. 8 is a the same view of the distal end of the shaft depicted in FIG. 3, except the lap joint circumferential surface has ridges that give the surface a barbed or ridged profile.

FIG. 9 is the same view of the distal end of the shaft depicted in FIG. 8, except the tip as been installed on the shaft and the shaft has been reduced to its final diameter.

### DETAILED DESCRIPTION

The present invention, in one embodiment, is a method of attaching or bonding a shaft tip to a catheter or sheath shaft. The method is advantageous because it is less labor intensive than prior art methods and results in less scrap. Throughout this specification, the term shaft is meant to include, without limitation, shafts for catheters, sheaths and similar medical equipment.

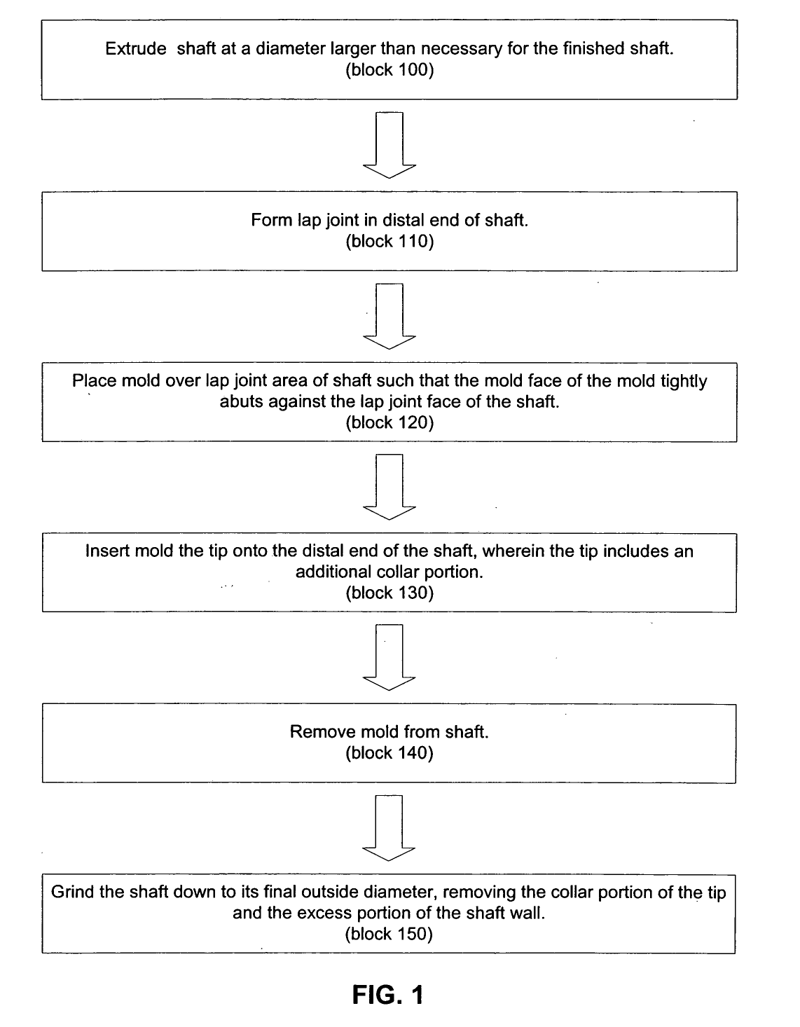

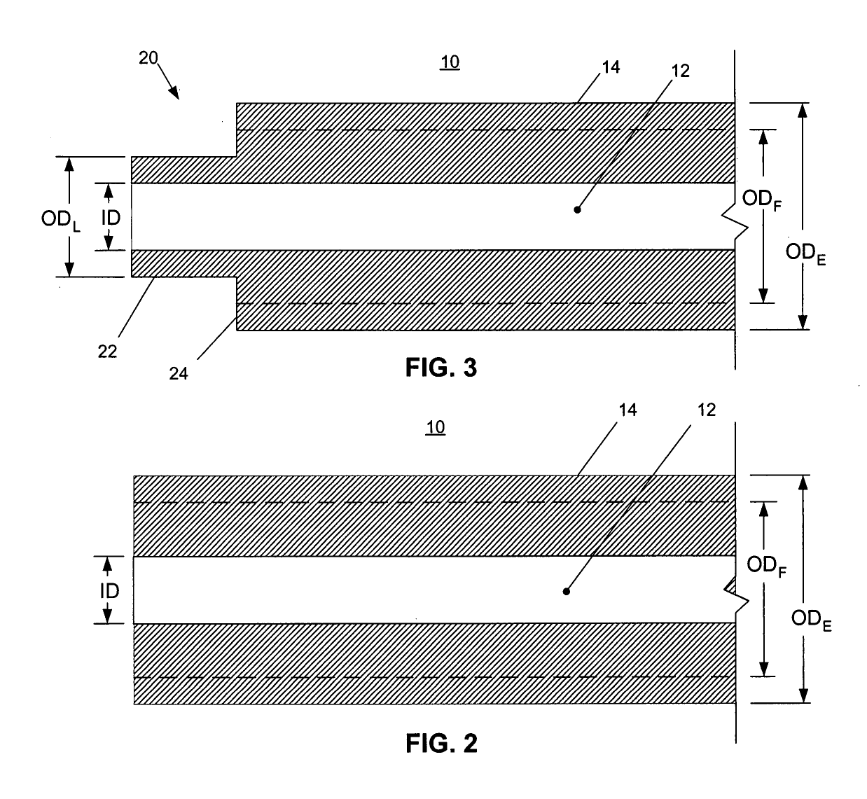

For a detailed discussion of one embodiment of the invention, reference is now made to FIGS. 1-5. FIG. 1 is a flow chart outlining the procedures comprising a method of attaching or bonding a shaft tip to a catheter or sheath shaft. FIGS. 2-5 are longitudinal sectional elevations of a distal end of a shaft 10 at the various stages of the manufacturing method, wherein the shaft 10 includes a central lumen 12 defined by a shaft sidewall 14.

As indicated in FIGS. 1 and 2, the shaft 10 is extruded such that the shaft's extruded outside diameter OD_{E} is larger than the shaft's finished outside diameter OD_{F}, which is represented in FIGS. 2-6 by dashed lines (block 100). In one embodiment, where the internal diameter ID is approximately 0.094″, the extruded outside diameter OD_{E} is approximately 0.155″ and the finished outside diameter OD_{F} is approximately 0.115″. In other words, in one embodiment, the extruded outside diameter OD_{E} is approximately 0.040″ larger than the finished outside diameter OD_{F}.

In one embodiment, the shaft 10 is formed of polyethylene, polyether block amides “PEBAX”, or other polymer materials. In one embodiment, the shaft is extruded. In other embodiments, the shaft 10 is comprised of multiple extruded polymer segments, metallic braid, lubricious liner components, and etc. that are reflowed into one contiguous component.

As shown in FIGS. 1 and 3, a lap joint 20 is ground into the distal end of the shaft 10 (block 110), thereby forming a lap joint circumferential surface 22 and a lap joint face 24. The lap joint circumferential surface 22 is generally parallel to the outer circumferential surface of the shaft sidewall 14, and the lap joint face 24 is generally perpendicular to the lap joint circumferential surface 22. In one embodiment, the grinding of the lap joint 20 results in the lap joint circumferential surface 22 having a lap joint outside diameter OD_{L} of approximately 0.102″. In other words, in one embodiment, the lap joint outside diameter OD_{L} is approximately 0.053″ smaller than the extruded outside diameter OD_{E} and approximately 0.008″ greater than the inside diameter ID.

In one embodiment, the lap joint 20 is ground via a centerless grinding system. In other embodiments, the lap joint is ground via a standard single wheel grinding system. In other embodiments, the lap joint is formed via a thermoforming process. In other embodiments, the lap joint is formed via laser material removal, chemical etching, mechanical machining (lathe), or water jet cutting.

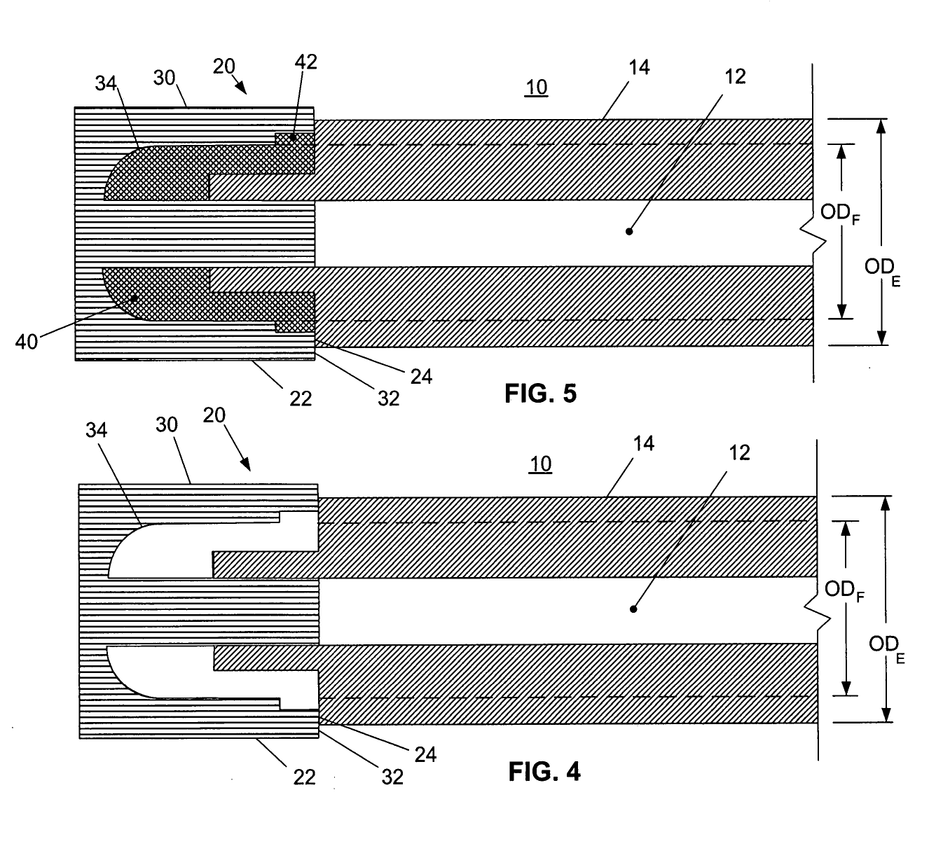

As indicated in FIGS. 1 and 4, a mold 30 is placed over the lap joint 20 of the distal end of the shaft 10 such that the mold face 32 tightly abuts against the lap joint face 24 of the shaft 10, and the mold interior 34 defines a void for forming a shaft tip (block 120). Because the grinding operations used to form the lap joint 20 offer much more precise tolerances as compared to the extrusion processes used to form the shaft 10, the shutoff formed between the lap joint face 24 and the mold face 32 is significantly tighter and less likely to result in flash than a shutoff formed between the outer circumferential surface of the shaft sidewall 14 and a corresponding mold. Also, because the point of contact between the mold face 32 and the lap joint face 24 is exterior to the finished outside diameter OD_{F}, any crimping or deforming of the sidewall 14 of the shaft 10 occurs in the portion of the sidewall 14 to be removed when the shaft 10 is reduced to its the finished outside diameter OD_{F}.

As shown in FIGS. 1 and 5, a shaft tip 40 is insert molded over the lap joint 20 area of the distal end of the shaft 10 and within the mold interior 34 (block 130). As shown in FIG. 5, in one embodiment, most of the tip 40 has an outside diameter generally equal to the finished outside diameter OD_{F}. In one embodiment, the tip 40 also includes a collar portion 42 that extends continuously about the outer circumferential surface of the tip 40. The collar portion 42 is advantageous in that it provides a great deal of additional thermal mass to the tip 40, thereby facilitating the forming of the tip/shaft bond.

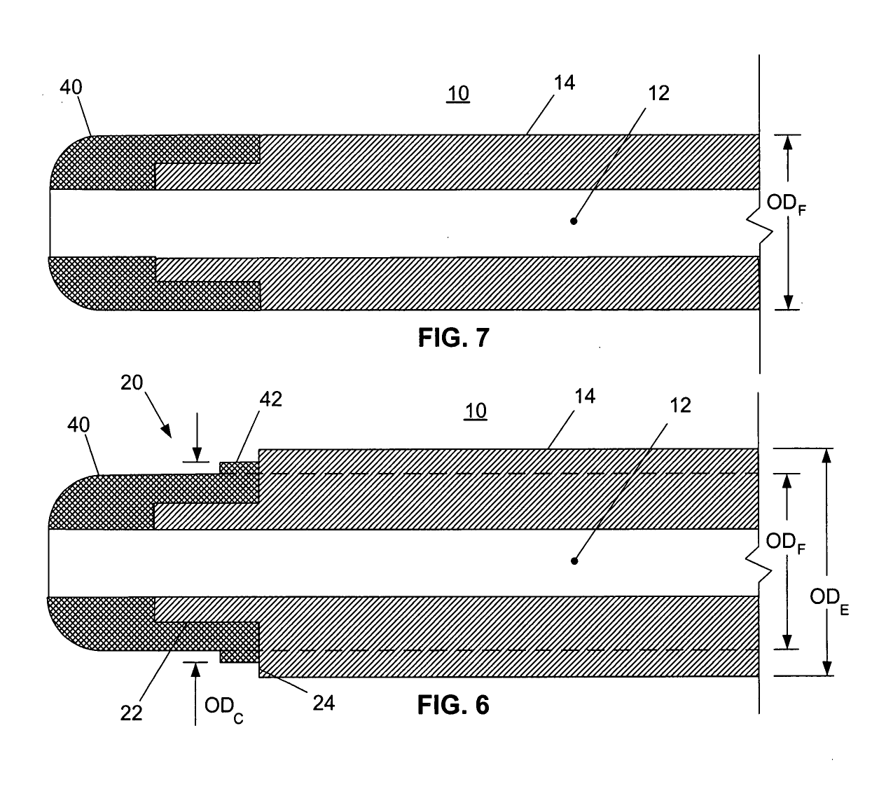

Once the shaft tip 40 has adequately cooled, the mold 30 is removed (block 140) and the distal end of the shaft 10 appears as indicated in FIG. 6. As can be appreciated from FIG. 6, the collar outside diameter OD_{C} is approximately midway in size between the extruded outside diameter OD_{E} and the finished outside diameter OD_{F}. In one embodiment, the collar outside diameter OD_{C} is at least approximately 0.005″ greater than the finished outside diameter OD_{F}. In other words, in one embodiment, the collar outside diameter OD_{C} is approximately 0.120″. In one embodiment, the shaft tip 40 and collar 42 are formed from polypropylene, santaprene molding resin, polyethylene, polyether block amides “PEBAX”, or other types and combinations of polymers.

As indicated in FIGS. 1 and 7, the shaft 10 is ground down to its finished outside diameter OD_{F}, (block 150) which, depending on the embodiment and the shaft's intended use, will be from approximately 0.013″ (1 French) to approximately 0.325″ (25 French). As can be understood from FIG. 7, in reducing the shaft 10 to its finished outside diameter OD_{F}, the shaft tip collar 42 and the excess portion of the shaft sidewall 14 are ground away. Thus, even if flash or another deformation occurs along the extruded outside diameter OD_{E} during the molding process, such unwanted defects can be ground away, thereby eliminating the need to scrap the shaft 10.

In one embodiment, the grinding process used to reduce the extruded outside diameter OD_{E} to the finished outside diameter OD_{F} is performed on a centerless grinder. In other embodiments, the grinding process is performed on other grinding systems, such as a standard single wheel grinding system.

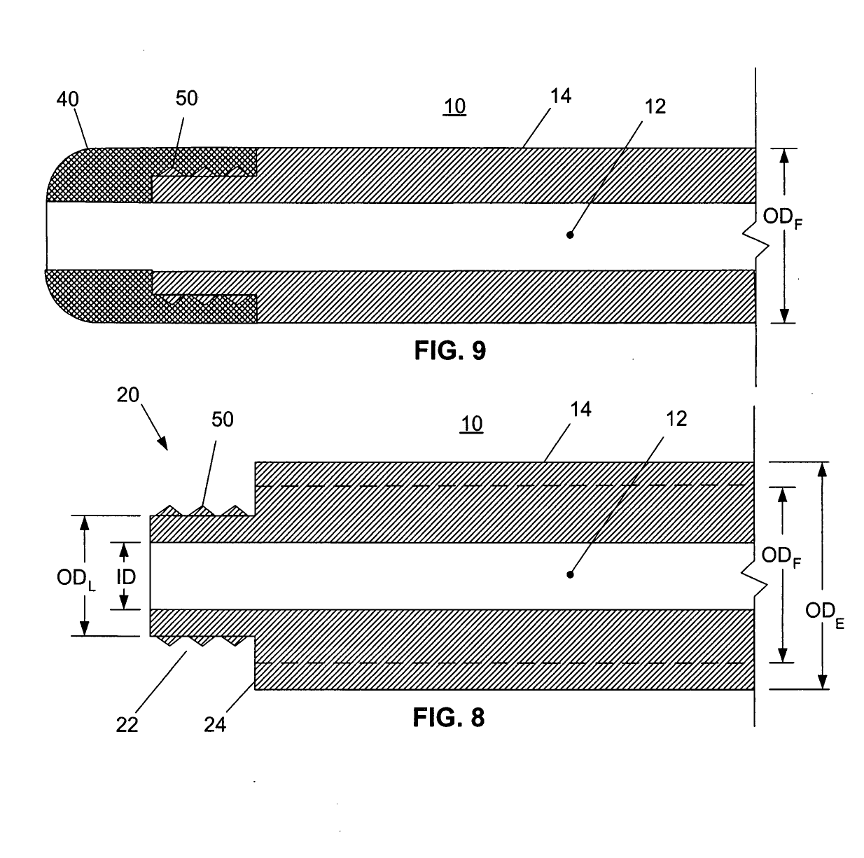

As indicated in FIGS. 3-7, in one embodiment, the lap joint circumferential surface 22 is formed such that it has a generally uniform linear profile. However, in other embodiments, the lap joint circumferential surface 22 is formed such that it has a non-linear profile. For example, as shown in FIG. 8, which is the same view of the distal end of the shaft depicted in FIG. 3, in one embodiment, the lap joint circumferential surface 22 is ground to have ridges 50 that give the surface 22 a barbed or ridged profile.

As indicated in FIG. 9, which is the same view of the distal end of the shaft 10 depicted in FIG. 8, except the tip as been installed on the shaft 10 and the shaft 10 has been reduced to its finished outside diameter OD_{F}, the ridges 50 serve as a mechanical feature for increasing the hold between the tip 40 and the lap joint circumferential surface 22. Thus, as indicated in FIG. 9, the tip 40 is attached to the lap joint circumferential surface 22 via the mechanical aspects of the ridges 50 and thermal bonding between the material of the tip 40 and the shaft sidewall 14.

Although the present invention has been described with reference to preferred embodiments, persons skilled in the art will recognize that changes may be made in form and detail without departing from the spirit and scope of the invention.

1. A tip for bonding to a distal end of a catheter or sheath shaft, the tip comprising:
a generally cylindrical first portion including a first outside diameter, a proximal end adapted to bond to the distal end of the shaft, and a distal end opposite the proximal end; and
a collar portion extending around a proximal portion of the first portion and including a second outside diameter that is greater than the first outside diameter.

2. The tip of claim 1, wherein the distal end of the shaft has a lap joint area and the tip is adapted to be bonded over the lap joint area.

3. The tip of claim 1, wherein the collar portion is adapted to abut against, and bond with, a lap joint face.

4. The tip of claim 1, wherein the second outside diameter is less than an extruded outside diameter of the shaft.

5. A method of bonding a tip to a distal end of a catheter or sheath shaft, the method comprising:
providing a shaft with an initial outside diameter;
forming a lap joint area in the distal end of the shaft;
molding the tip over the lap joint area; and
reducing the initial outside diameter of the shaft down to a finished outside diameter.

6. The method of claim 5, wherein the tip includes a collar at a proximal end of the tip.

7. The method of claim 6, wherein the collar has an outside diameter that is greater than the finished outside diameter.

8. The method of claim 7, wherein the outside diameter of the collar is less than the initial outside diameter of the shaft.

9. The method of claim 5, further comprising forming a point of contact between a mold face and a lap face of the lap joint area such that the point of contact is outside the finished outside diameter.

10. The method of claim 5, wherein the initial outside diameter is reduced down to the finished outside diameter by a grinding process.

11. The method of claim 10, wherein the grinding process occurs on a centerless grinder.

12. The method of claim 5, wherein the lap joint area is formed via a grinding process.

13. The method of claim 5, wherein the tip is molded over the lap joint area via an insert molding process.

14. The method of claim 5, wherein lap joint area has a non-linear profile.

15. The method of claim 14, wherein the non-linear profile is the result of a ridge being formed in the lap joint area.

16. A catheter or sheath shaft made according to method of claim 5.

17. A method of bonding a tip to a distal end of a catheter or sheath shaft, the method comprising:
forming an oversized shaft having an oversized outside diameter;
molding a tip onto a distal end of the oversized shaft; and
converting the oversized shaft into a finished shaft by reducing the oversized outside diameter to a finished outside diameter.

18. The method of claim 17, wherein the oversized shaft is formed via an extrusion process.

19. The method of claim 17, wherein the tip is molded onto the distal end via an insert molding process.

20. The method of claim 17, wherein the oversized outside diameter is reduced to the finished outside diameter via a grinding process.

21. The method of claim 17, further comprising forming a lap joint in the distal end of the oversized shaft for receiving the tip during the molding process.

22. The method of claim 21, further comprising forming a ridge in the lap joint such that the lap joint has a non-linear profile.

23. The method of claim 21, wherein the lap joint is formed via a grinding process.

24. The method of claim 17, wherein the tip includes a collar portion extending circumferentially about a proximal portion of the tip.
